(19) United States
(12) Patent Application Publication
(10) Pub. No.: US 2021/0322288 A1
(43) Pub. Date: Oct. 21, 2021

(54) Toothpaste Compositions Comprising Silica And Surface Active Polymer

(71) Applicant: The Procter & Gamble Company, Cincinnati, OH (US)

(72) Inventors: Larissa Azirbayeva, Mason, OH (US); Jessica Ong Meza, Cincinnati, OH (US); Marco Caggioni, Cincinnati, OH (US); Matthew Frazier Laird, Cincinnati, OH (US); Yuri Volodymyrovych Roiter, Loveland, OH (US); Christopher Medley, Cincinnati, OH (US); Fang Hu, Mason, OH (US); William Handy Hartt, Mason, OH (US); Setareh Shahsavari, Cincinnati, OH (US)

(21) Appl. No.: 17/233,579

(22) Filed: Apr. 19, 2021

Related U.S. Application Data
(60) Provisional application No. 63/034,420, filed on Jun. 4, 2020.
(60) Provisional application No. 63/012,419, filed on Apr. 20, 2020.

(51) Int. Cl. A61K 8/24; A61K 8/25; A61K 8/81; A61K 8/22; A61K 8/21; A61K 8/27; A61K 8/19; A61Q 11/00

(52) U.S. Cl. CPC A61K 8/24; A61K 8/25; A61K 8/8182; A61K 8/22; A61K 2800/412; A61K 8/27; A61K 8/19; A61Q 11/00; A61K 2800/28; A61K 8/21

(57) ABSTRACT

An oral care composition with silica and polyvinylpyrrolidone showing an increased in viscosity after a shear event, viscosity recovery, of at least about 1.5× within approximately 348 seconds. A low-water oral care composition with improved rheology. A low-water oral care composition that does not leak from a toothpaste tube if inverted. An oral care composition with low water, silica, surface active polymer, and optionally polyphosphate.

### FIELD OF THE INVENTION

The present invention relates to oral care compositions comprising silica and surface active polymer. The present invention also relates to oral care compositions with improved rheology, such as viscosity recovery after a shear event. The present invention also relates to oral care compositions that demonstrate low or no leakage from a tube.

### BACKGROUND OF THE INVENTION

Polyphosphates, or compounds with more than one phosphate groups bound either linearly or in a cyclic arrangement, are a common ingredient in oral care compositions. Polyphosphates can remove stains from teeth. However, longer chain polyphosphate molecules, such as polyphosphates with greater than three phosphate groups, can also be susceptible to hydrolysis in oral care compositions comprising water, such as greater than 10% of water. Thus, many oral care compositions comprising linear polyphosphates, such as hexametaphosphate, can be formulated as low or lower water formulas. Lowering the amount of water in an oral care composition, such as a toothpaste and/or dentifrice, can limit reactivity between components in oral care compositions.

Unfortunately, while lower water formulations prevent reactivity in the tube, such as between polyphosphate and water and/or between tin and silica, lower water formulations can be difficult to thicken. As a result, many lower or no added water dentifrice and/or toothpaste composition can be runny or leak out of the tube, especially after dispensing. Thus, there is a need for low-water toothpaste composition with improved viscosity recovery upon dispensing.

### SUMMARY OF THE INVENTION

Disclosed herein is a toothpaste composition comprising (a) from about 0.01% to about 5%, by weight of the composition, of surface active polymer; (b) from about 0.1% to about 40%, by weight of the composition of abrasive, the abrasive comprising silica; and (c) less than 10%, by weight of the composition, of water, wherein the toothpaste composition has a ratio of surface active polymer to silica surface area of from about 0.01 to about 2.0 mg/m².

Also disclosed herein is a toothpaste composition comprising (a) from about 0.01% to about 5%, by weight of the composition, of surface active polymer; (b) from about 0.1% to about 40%, by weight of the composition of abrasive, the abrasive comprising silica; and (c) less than 10%, by weight of the composition, of water, wherein the toothpaste composition has a first viscosity measured less than 5 seconds after a shear event and the toothpaste composition has a second viscosity measured at least 50 seconds after the shear event, and the second viscosity is greater the first viscosity.

Also disclosed herein is a toothpaste composition comprising (a) from about 0.01% to about 5%, by weight of the composition, of surface active polymer, preferably wherein the surface active polymer comprises polyvinylpyrrolidone, cross-linked polyvinylpyrrolidone, another polymer derived from a N-vinylpyrrolidone monomer, polyethylene oxide, poloxamer, or combinations thereof; (b) from about 0.1% to about 40%, by weight of the composition of abrasive, the abrasive comprising silica, preferably wherein the silica comprises abrasive silica, thickening silica, or combinations thereof; and (c) less than 10%, by weight of the composition, of water, wherein the toothpaste composition has a ratio of surface active polymer to silica of from about 0.01 to about 2.0 mg/m², preferably wherein the toothpaste composition has a ratio of surface active polymer to silica of from about 0.05 to about 0.80 mg/m².

### BRIEF DESCRIPTION OF THE DRAWINGS

FIG. 1 shows the viscosity recovery of a toothpaste comprising thickening silica and polyvinylpyrrolidine.

FIG. 2 shows the viscosity recovery of a toothpaste comprising abrasive silica and polyvinylpyrrolidine.

### DETAILED DESCRIPTION OF THE INVENTION

The present invention is directed to low water or anhydrous oral care compositions, such as toothpaste compositions, with improved rheology and/or demonstrate low or no leakage from the tube. Typically, lower or no water oral care compositions can be thickened by increasing the amount of thickening agent. For example, increasing the amount of thickening agent can increase the yield stress (i.e. the force required to start movement of the composition within the dispenser). However, increasing the yield stress by increasing the amount of thickening agent, without a corresponding decrease in shear index (lowering Power Law “n” to make it more shear thinning), will result in increases the viscosity of the composition when it is being dispensed, thereby making the composition more difficult to dispense. In other words, increasing the amount of thickening material can make the compositions stored within a tube, less likely to leak, but harder to dispense. In contrast, desired herein, are compositions that remain easy to dispense, very shear thinning, but without any leakage from tubes due to fast viscosity recovery after a shear event.

Unexpectedly, it has been found that compositions comprising particular ratios of certain surface active polymers, such as polyvinylpyrrolidone (PVP), and silica, can have viscosity recovery after dispensed from a tube. Compositions that display viscosity recovery after subjected to a shear event, such as dispensing from a tube, can lead to compositions that remain easy to dispense, but are less likely to leak from the tube during storage. Importantly, these unexpected results only occurred within a targeted range of mg surface active polymer to available surface area of silica. While not wishing to being bound by theory, it is believed that these unexpected results occurred because the surface active polymer, such as PVP, reacts with the silica surface, forming a corona, or other supramolecular structure, around the silica particle potentially leading to steric stabilization or bridging across silica particles. Thus, this unique toothpaste rheology is present when the silica surface area is at least partially coated with surface active polymers, such as PVP.

### Definitions

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), can be applied, as long as that definition does not conflict with any other disclosure or definition applied herein, or render indefinite or non-enabled any claim to which that definition is applied.

The term “oral care composition”, as used herein, includes a product, which in the ordinary course of usage, is not intentionally swallowed for purposes of systemic administration of particular therapeutic agents, but is rather retained in the oral cavity for a time sufficient to contact dental surfaces or oral tissues. Examples of oral care compositions include dentifrice, toothpaste, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The oral care composition may also be incorporated onto strips or films for direct application or attachment to oral surfaces.

The term “dentifrice composition”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

“Active and other ingredients” useful herein may be categorized or described herein by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated function(s) or activities listed.

The term “orally acceptable carrier” comprises one or more compatible solid or liquid excipients or diluents which are suitable for topical oral administration. By “compatible,” as used herein, is meant that the components of the composition are capable of being commingled without interaction in a manner which would substantially reduce the composition's stability and/or efficacy. The carriers or excipients of the present invention can include the usual and conventional components of mouthwashes or mouth rinses, as more fully described hereinafter: Mouthwash or mouth rinse carrier materials typically include, but are not limited to one or more of water, alcohol, humectants, surfactants, and acceptance improving agents, such as flavoring, sweetening, coloring and/or cooling agents.

The term “substantially free” as used herein refers to the presence of no more than 0.05%, preferably no more than 0.01%, and more preferably no more than 0.001%, of an indicated material in a composition, by total weight of such composition.

The term “essentially free” as used herein means that the indicated material is not deliberately added to the composition, or preferably not present at analytically detectable levels. It is meant to include compositions whereby the indicated material is present only as an impurity of one of the other materials deliberately added.

While compositions and methods are described herein in terms of “comprising” various components or steps, the compositions and methods can also “consist essentially of” or “consist of” the various components or steps, unless stated otherwise.

As used herein, the word “or” when used as a connector of two or more elements is meant to include the elements individually and in combination; for example, X or Y, means X or Y or both.

As used herein, the articles “a” and “an” are understood to mean one or more of the material that is claimed or described, for example, “an oral care composition” or “a bleaching agent.”

All measurements referred to herein are made at about 23° C. (i.e. room temperature) unless otherwise specified.

Generally, groups of elements are indicated using the numbering scheme indicated in the version of the periodic table of elements published in Chemical and Engineering News, 63(5), 27, 1985. In some instances, a group of elements can be indicated using a common name assigned to the group; for example, alkali metals for Group 1 elements, alkaline earth metals for Group 2 elements, and so forth.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein.

The term “about” means that amounts, sizes, formulations, parameters, and other quantities and characteristics are not and need not be exact, but can be approximate and/or larger or smaller, as desired, reflecting tolerances, conversion factors, rounding off, measurement errors, and the like, and other factors known to those of skill in the art. In general, an amount, size, formulation, parameter or other quantity or characteristic is “about” or “approximate” whether or not expressly stated to be such. The term “about” also encompasses amounts that differ due to different equilibrium conditions for a composition resulting from a particular initial mixture. Whether or not modified by the term “about,” the claims include equivalents to the quantities. The term “about” can mean within 10% of the reported numerical value, preferably within 5% of the reported numerical value.

The oral care composition can be in any suitable form, such as a solid, liquid, powder, paste, or combinations thereof. The oral care composition can be dentifrice, toothpaste, tooth gel, subgingival gel, mouth rinse, mousse, foam, mouth spray, lozenge, chewable tablet, chewing gum, tooth whitening strips, floss and floss coatings, breath freshening dissolvable strips, or denture care or adhesive product. The components of the oral care composition can be incorporated into a film, a strip, a foam, or a soluble fiber-based oral care composition. The oral care composition can include a variety of active and inactive ingredients, such as, for example, but not limited to silica, surface active polymer, lower or no water, polyphosphate, abrasive, fluoride, tin, zinc, humectants, surfactants, whitening agents, other ingredients, and the like, as well as any combination thereof, as described below.

While the specification concludes with claims particularly pointing out and distinctly claiming the invention, it is believed that the present invention will be better understood from the following description.

### Silica

The oral care composition of the present invention comprises silica. The silica can comprise abrasive silica, thickening silica, or combinations thereof.

Silica can be described by its level of abrasivity of the particles of silica. Non-abrasive or low abrasive silica can also be known as thickening silica. Silica particles can have particles with a median particle size of up to about 12 μm (high abrasive silica), from about 12 μm to about 14 μm (low abrasive silica), greater than about 14 μm (non-abrasive silica) or, in total, up to about 20 μm. The method for determining the median particle size of silica particles is well-known by a person of ordinary skill in the art.

By BET (Brunauer, Emmett and Teller) the specific surface area of a sample is measured—including the pore size distribution. This information is used to predict the dissolution rate, as this rate is proportional to the specific surface area. Thus, the surface area can be used to predict bioavailability. Further it is useful in evaluation of product performance and manufacturing consistency.

The specific surface determined by BET relates to the total surface area (reactive surface) as all porous structures adsorb the small gas molecules. The method used complies with Ph. Eu.2.9.26 Method II.

Other suitable silica particles include silica particles with a BET surface area of from about 10 m²/g to about 600 m²/g, from about 20 m²/g to about 30 m²/g (highly abrasive silica), from about 60 m²/g to about 90 m²/g (low abrasive silica), from about 200 m²/g to about 600 m²/g (non-abrasive silica), from about 20 m²/g to about 200 m²/g, or from about 20 m²/g to about 600 m²/g. The method for determining the BET surface area of silica particles is well-known by a person of ordinary skill in the art.

The silica of the present invention can comprise silica abrasive. Silica abrasives can be added to oral care formulations to help remove surface stains from teeth.

The oral care composition can also comprise a silica abrasive, such as silica gel (by itself, and of any structure), precipitated silica, amorphous precipitated silica (by itself, and of any structure as well), hydrated silica, and/or combinations thereof. The oral care composition can comprise from about 5% to about 70%, from about 5% to about 40%, from about 10% to about 60%, from about 10% to about 50%, from about 20% to about 50%, from about 25% to about 40%, or from about 1% to about 50%, by weight of the oral care composition, of silica abrasive.

The oral care composition can also comprise thickening silica. Suitable silica thickeners include, for example, include, as a non-limiting example, an amorphous precipitated silica such as ZEODENT® 165 silica. Other non-limiting silica thickeners include ZEODENT® 153, 163, and 167, and ZEOFREE® 177 and 265 silica products, all available from Evonik Corporation, and AEROSIL® fumed silicas. The oral care composition can comprise from about 0.01% to about 20%, from about 1% to about 10%, from about 0.1% to about 15%, or from about 1% to about 25%, by weight of the oral care composition of thickening silica.

In total, the oral care composition can comprise from about 0.01% to about 70%, from about 0.1% to about 40%, or from about 1% to about 25%, by weight of the oral care composition, of silica.

### Surface Active Polymer

The oral care composition of the present invention comprises surface active polymer. The surface active polymer can interact with the surface of silica to provide viscosity recovery after dispensing from an oral care composition tube.

The polymer can comprise polyvinylpyrrolidone (PVP), cross-linked polyvinylpyrrolidone, another polymer derived from a N-vinylpyrrolidone monomer, polyethylene oxide, poloxamer, or combinations thereof.

The oral care composition can comprise from about 0.01% to about 15%, from about 0.01% to about 10%, from about 0.01% to about 2%, from about 0.01% to about 5%, from about 0.05% to less than 2%, from about 0.05% to about 1.5%, from about 0.05% to about 1%, or from about 0.05 to about 5%, by weight of the composition, of the surface active polymer, such as PVP.

A suitable amount of the surface active polymer can be described by the ratio of the amount of surface active polymer to the total surface area provided by silica particles in the oral care composition. Suitable ratios include from about 0.01 to about 3, from about 0.01 to about 2, from about 0.01 to about 1.5, from about 0.01 to about 1, from about 0.05 to about 0.8, from about 0.05 to about 0.6, or from about 0.1 to about 0.5 mg/m².

While not wishing to being bound by theory, it is believed that the ideal rheological benefits (i.e. the viscosity recovery after a shear event) is believed to observed only at a specific ratio of amount of surface active polymer, such as PVP, to the silica surface area, as described herein. The ratios described herein likely refer to when at least a portion of the surface area of silica is associated with the surface active polymer. As the silica surface area begins to be fully associated with surface active polymer to create supramolecular structures that can provide viscosity in the toothpaste composition. Beyond this point, additional amounts are not likely beneficial. While not wishing to being bound by theory, it is believed that the supramolecular structure provided by the surface active polymer adsorbed to silica provided the unique viscosity recovery behavior unlike other thickening agents which can only increase the viscosity in total, which can also lead to detrimental effects, such as increased difficulty dispensing the toothpaste composition.

### Water

The oral care composition can be anhydrous or a low water formulation. In total, the oral care composition, of the present invention comprises from 0% to about 25%, from 0% to about 10%, up to about 10%, less than 10%, less than 5%, or less than 1%, by weight of the composition, of water. The water may be added to the formulation and/or may come into the oral care composition from the inclusion of other ingredients. Preferably, the water, if added, is USP water. A lower or no water formulation is preferred in certain oral care compositions to prevent and/or mitigate reactivity, between ingredients within the oral care compositions, such as water and polyphosphate, tin and silica, etc.

### Polyphosphate

The dentifrice composition can comprise polyphosphate. Polyphosphate can be provided by a polyphosphate source. A polyphosphate source can comprise one or more polyphosphate molecules. Polyphosphates are a class of materials obtained by the dehydration and condensation of orthophosphate to yield linear and cyclic polyphosphates of varying chain lengths. Thus, polyphosphate molecules are generally identified with an average number (n) of polyphosphate molecules, as described below. A polyphosphate is generally understood to consist of two or more phosphate molecules arranged primarily in a linear configuration, although some cyclic derivatives may be present.

Preferred polyphosphates include those having an average of four or more phosphate groups so that surface adsorption at effective concentrations produces sufficient non-bound phosphate functions, which enhance the anionic surface charge as well as hydrophilic character of the surfaces. Preferred in this invention are the linear polyphosphates having the formula: XO(XPO₃)ₙX, wherein X is sodium, potassium, ammonium, or any other alkali metal cations and n averages from about 4 to about 21. Alkali earth metal cations, such as calcium, are not preferred because they tend to form insoluble fluoride salts from aqueous solutions comprising a fluoride ions and alkali earth metal cations. Thus, the dentifrice compositions disclosed herein can be free of or substantially free of calcium pyrophosphate.

Some examples of suitable polyphosphate molecules include, for example, tetraphosphate (n=4), sodaphos polyphosphate (n=6), hexaphos polyphosphate (n=13), benephos polyphosphate (n=14), hexametaphosphate (n=21), which is also known as Glass H. Polyphosphates can include those polyphosphate compounds manufactured by FMC Corporation, ICL Performance Products, and/or Astaris.

The oral care composition can comprise from about 0.01% to about 15%, from about 0.1% to about 10%, from about 0.5% to about 5%, from about 1 to about 20%, or about 10% or less, by weight of the composition, of polyphosphate.

### Additional Abrasive

The oral care composition can comprise from about 0.5% to about 75% of an additional abrasive by weight of the oral care composition. The oral care composition can comprise from about 5% to about 60%, from about 10% to about 50%, or from about 15% to about 55%, or combinations thereof, of the additional abrasive by weight of the composition. The additional abrasive can be a calcium-containing abrasive, a carbonate abrasive, a phosphate abrasive, an alumina abrasive, other suitable abrasives, and/or combinations thereof. Some abrasives may fit into several descriptive categories, such as for example calcium carbonate, which is both a calcium-containing abrasive and a carbonate abrasive.

The calcium-containing abrasive can comprise calcium carbonate, dicalcium phosphate, tricalcium phosphate, calcium orthophosphate, calcium metaphosphate, calcium polyphosphate, calcium hydroxyapatite, and combinations thereof.

The calcium-containing abrasive can comprise calcium carbonate. The calcium-containing abrasive can be selected from the group consisting of fine ground natural chalk, ground calcium carbonate, precipitated calcium carbonate, and combinations thereof.

The carbonate abrasive can comprise sodium carbonate, sodium bicarbonate, calcium carbonate, strontium carbonate, and/or combinations thereof.

The phosphate abrasive can comprise calcium phosphate, sodium hexametaphosphate, dicalcium phosphate, tricalcium phosphate, calcium orthophosphate, calcium metaphosphate, calcium polyphosphate, a polyphosphate, a pyrophosphate, and/or combinations thereof.

The alumina abrasive can comprise polycrystalline alumina, calcined alumina, fused alumina, levigated alumina, hydrated alumina, and/or combinations thereof.

Other suitable abrasives include diatomaceous earth, barium sulfate, wollastonite, perlite, polymethylmethacrylate particles, tospearl, and combinations thereof.

### Fluoride

The dentifrice composition can comprise fluoride. Fluoride can be provided by a fluoride ion source. The fluoride ion source can comprise one or more fluoride containing compounds, such as stannous fluoride, sodium fluoride, potassium fluoride, amine fluoride, sodium monofluorophosphate, zinc fluoride, and/or mixtures thereof.

The fluoride ion source and the tin ion source can be the same compound, such as for example, stannous fluoride, which can generate tin ions and fluoride ions. Additionally, the fluoride ion source and the tin ion source can be separate compounds, such as when the tin ion source is stannous chloride and the fluoride ion source is sodium monofluorophosphate or sodium fluoride.

The fluoride ion source and the zinc ion source can be the same compound, such as for example, zinc fluoride, which can generate zinc ions and fluoride ions. Additionally, the fluoride ion source and the zinc ion source can be separate compounds, such as when the zinc ion source is zinc phosphate and the fluoride ion source is stannous fluoride.

The dentifrice composition can comprise a fluoride ion source capable of providing from about 50 ppm to about 5000 ppm, and preferably from about 500 ppm to about 3000 ppm of free fluoride ions. The oral care composition can comprise from about 0.001% to about 10%, 0.01% to about 5%, from about 0.1 to about 2%, or from about 0.5% to about 1.5%, by weight of the oral care composition, of fluoride.

### Tin

The oral care composition of the present invention can comprise tin. The tin can be provided by a tin ion source. The tin ion source can be any suitable compound that can provide tin ions in a dentifrice composition and/or deliver tin ions to the oral cavity when the dentifrice composition is applied to the oral cavity. The tin ion source can comprise one or more tin containing compounds, such as stannous fluoride, stannous chloride, stannous bromide, stannous iodide, stannous oxide, stannous oxalate, stannous sulfate, stannous sulfide, stannic fluoride, stannic chloride, stannic bromide, stannic iodide, stannic sulfide, and/or mixtures thereof. Preferably, the tin ion source can comprise stannous fluoride, stannous chloride, and/or mixture thereof.

The oral care composition can comprise from about 0.001% to about 10%, 0.01% to about 5%, from about 0.1 to about 2%, or from about 0.5% to about 1.5%, by weight of the oral care composition, of tin.

### Zinc

The oral care composition can comprise zinc. The zinc can be provided by a zinc ion source. The zinc ion source can comprise one or more zinc containing compounds, such as zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, and/or zinc carbonate.

The oral care composition can comprise from about 0.001% to about 10%, 0.01% to about 5%, from about 0.1 to about 2%, or from about 0.5% to about 1.5%, by weight of the oral care composition, of zinc.

### Humectant

The oral care composition can comprise a humectant, have low levels of a humectant, or be free of a humectant. Humectants serve to add body or “mouth texture” to an oral care composition or dentifrice as well as preventing the dentifrice from drying out. Suitable humectants include polyethylene glycol (at a variety of different molecular weights), propylene glycol, glycerin (glycerol), erythritol, xylitol, sorbitol, mannitol, butylene glycol, lactitol, hydrogenated starch hydrolysates, and/or mixtures thereof. The oral care composition can comprise one or more humectants each at a level of from 0 to about 70%, from about 5% to about 50%, from about 10% to about 60%, or from about 20% to about 80%, by weight of the oral care composition.

### Surfactant

The oral care composition can comprise one or more surfactants. The surfactants can be used to make the compositions more cosmetically acceptable. The surfactant is preferably a detersive material which imparts to the composition detersive and foaming properties. Suitable surfactants are safe and effective amounts of anionic, cationic, nonionic, zwitterionic, amphoteric and betaine surfactants, such as sodium lauryl sulfate, sodium lauryl isethionate, sodium lauroyl methyl isethionate, sodium cocoyl glutamate, sodium dodecyl benzene sulfonate, alkali metal or ammonium salts of lauroyl sarcosinate, myristoyl sarcosinate, palmitoyl sarcosinate, stearoyl sarcosinate and oleoyl sarcosinate, polyoxyethylene sorbitan monostearate, isostearate and laurate, sodium lauryl sulfoacetate, N-lauroyl sarcosine, the sodium, potassium, and ethanolamine salts of N-lauroyl, N-myristoyl, or N-palmitoyl sarcosine, polyethylene oxide condensates of alkyl phenols, cocoamidopropyl betaine, lauramidopropyl betaine, palmityl betaine, sodium cocoyl glutamate, and the like. Sodium lauryl sulfate is a preferred surfactant. The oral care composition can comprise one or more surfactants each at a level from about 0.01% to about 15%, from about 0.3% to about 10%, or from about 0.3% to about 2.5%, by weight of the oral care composition.

### Whitening Agent

The oral care composition may comprise from about 0.1% to about 10%, from about 0.2% to about 5%, from about 1% to about 5%, or from about 1% to about 15%, by weight of the oral care composition, of a whitening agent. The whitening agent can be a compound suitable for whitening at least one tooth in the oral cavity. The whitening agent may include peroxides, metal chlorites, perborates, percarbonates, peroxyacids, persulfates, and combinations thereof. Suitable peroxides include solid peroxides, hydrogen peroxide, urea peroxide, calcium peroxide, benzoyl peroxide, sodium peroxide, barium peroxide, inorganic peroxides, hydroperoxides, organic peroxides, and mixtures thereof. Suitable metal chlorites include calcium chlorite, barium chlorite, magnesium chlorite, lithium chlorite, sodium chlorite, and potassium chlorite. Other suitable whitening agents include sodium persulfate, potassium persulfate, peroxydone, 6-phthalimido peroxy hexanoic acid, Pthalamidoperoxycaproic acid, or mixtures thereof.

### Additional Thickening Agents

In addition to the surface active polymer, the oral care composition can comprise additional thickening agents. Additional thickening agents can be useful in the dentifrice compositions to provide a gelatinous structure that stabilizes the toothpaste against phase separation. Suitable addotopma; thickening agents include polysaccharides, and/or polymers. Some non-limiting examples of polysaccharides include starch; glycerite of starch; gums such as gum karaya (sterculia gum), gum tragacanth, gum arabic, gum ghatti, gum acacia, xanthan gum, guar gum and cellulose gum; magnesium aluminum silicate (Veegum); carrageenan; sodium alginate; agar-agar; pectin; gelatin; cellulose compounds such as cellulose, carboxymethyl cellulose, hydroxyethyl cellulose, hydroxypropyl cellulose, hydroxymethyl cellulose, hydroxymethyl carboxypropyl cellulose, methyl cellulose, ethyl cellulose, and sulfated cellulose; natural and synthetic clays such as hectorite clays; and mixtures thereof.

The additional thickening agent can comprise polysaccharides. Polysaccharides that are suitable for use herein include carageenans, gellan gum, locust bean gum, xanthan gum, carbomers, poloxamers, modified cellulose, and mixtures thereof. Carageenan is a polysaccharide derived from seaweed. There are several types of carageenan that may be distinguished by their seaweed source and/or by their degree of and position of sulfation. The thickening agent can comprise kappa carageenans, modified kappa carageenans, iota carageenans, modified iota carageenans, lambda carrageenan, and mixtures thereof. Carageenans suitable for use herein include those commercially available from the FMC Company under the series designation “Viscarin,” including but not limited to Viscarin TP 329, Viscarin TP 388, and Viscarin TP 389.

The additional thickening agent can comprise one or more polymers. The polymer can be a polyethylene glycol (PEG), polyacrylic acid, a polymer derived from at least one acrylic acid monomer, a copolymer of maleic anhydride and methyl vinyl ether, a crosslinked polyacrylic acid polymer, of various weight percentages of the dentifrice composition as well as various ranges of average molecular ranges.

The oral care composition can comprise from 0.01% to about 15%, from 0.1% to about 10%, from about 0.2% to about 5%, or from about 0.5% to about 2%, by weight of the composition, of one or more thickening agents.

### Other Ingredients

The oral care composition can comprise a variety of other ingredients, such as flavoring agents, sweeteners, colorants, preservatives, buffering agents, or other ingredients suitable for use in oral care compositions, as described below.

Flavoring agents also can be added to the oral care composition. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. Coolants may also be part of the flavor system. Preferred coolants in the present compositions are the paramenthan carboxyamide agents such as N-ethyl-p-menthan-3-carboxamide (known commercially as “WS-3”) or N-(Ethoxycarbonylmethyl)-3-p-menthanecarboxamide (known commercially as “WS-5”), and mixtures thereof. A flavor system is generally used in the compositions at levels of from about 0.001% to about 5%, by weight of the oral care composition. These flavoring agents generally comprise mixtures of aldehydes, ketones, esters, phenols, acids, and aliphatic, aromatic and other alcohols.

Sweeteners can be added to the oral care composition to impart a pleasing taste to the product. Suitable sweeteners include saccharin (as sodium, potassium or calcium saccharin), cyclamate (as a sodium, potassium or calcium salt), acesulfame-K, thaumatin, neohesperidin dihydrochalcone, ammoniated glycyrrhizin, dextrose, levulose, sucrose, mannose, sucralose, stevia, and glucose.

Colorants can be added to improve the aesthetic appearance of the product. Suitable colorants include without limitation those colorants approved by appropriate regulatory bodies such as the FDA and those listed in the European Food and Pharmaceutical Directives and include pigments, such as TiO₂, and colors such as FD&C and D&C dyes.

Preservatives also can be added to the oral care compositions to prevent bacterial growth. Suitable preservatives approved for use in oral compositions such as methylparaben, propylparaben, benzoic acid, and sodium benzoate can be added in safe and effective amounts.

Titanium dioxide may also be added to the present composition. Titanium dioxide is a white powder which adds opacity to the compositions. Titanium dioxide generally comprises from about 0.25% to about 5%, by weight of the oral care composition.

Other ingredients can be used in the oral care composition, such as desensitizing agents, healing agents, caries preventative agents, chelating/sequestering agents, vitamins, amino acids, proteins, other anti-plaque/anti-calculus agents, opacifiers, antibiotics, anti-enzymes, enzymes, pH control agents, oxidizing agents, antioxidants, and the like.

### Examples

The invention is further illustrated by the following examples, which are not to be construed in any way as imposing limitations to the scope of this invention. Various other aspects, modifications, and equivalents thereof which, after reading the description herein, may suggest themselves to one of ordinary skill in the art without departing from the spirit of the present invention or the scope of the appended claims.

### Creep Test

The thixotropic behavior of a variety of toothpastes was determined by the Creep Test. The thixotropic behavior as described herein, was the ability of the toothpaste composition to recover high value of yield stress and shear viscosity after the application of shear, which simulated a dispensing event.

The Creep Test was developed to show how quickly the paste builds viscosity after a shear event over the course of 5 minutes. In the method, 35 mL of sample were first loaded in a 30 mm diameter cup geometry (TA instruments, New Castle, Del.). Once the sample was loaded, a vane geometry with diameter 28 mm (TA instrument, New Castle, Del.) was lowered into the sample down to a 6 mm operating gap from the bottom of the cup. Once the geometry was in place the vane was rotated at constant speed to maintain a shear rate of 40 s⁻¹ on the sample until the stress required to maintain the constant shear rate reached a steady state defined as having a variation <5% for 2 consecutive intervals of 5 seconds. The steady state was normally achieved within 1-2 minutes.

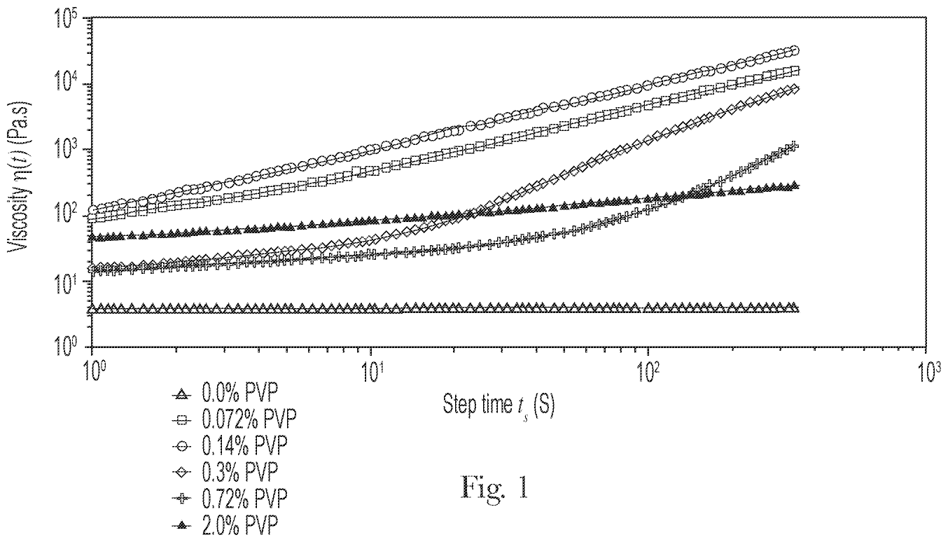

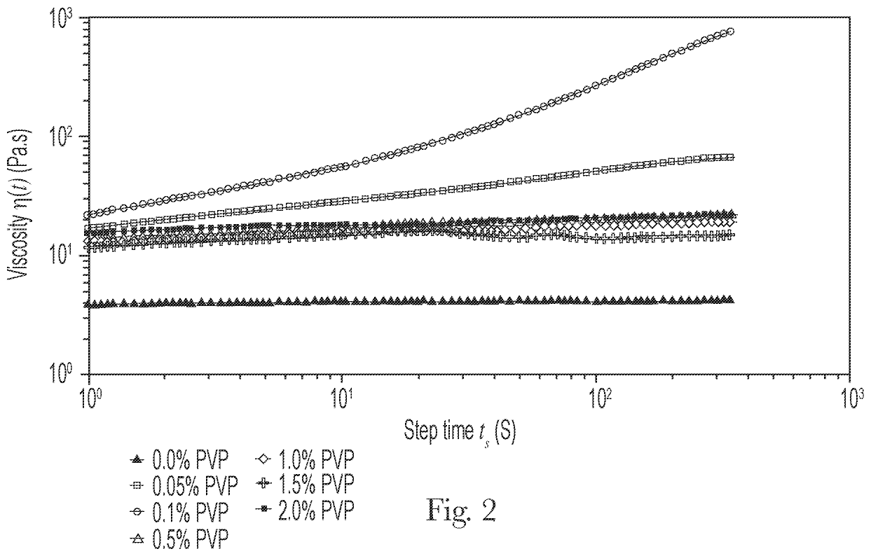

The sample was then allowed to rest for a max of ˜1 s before a constant stress of 20 Pa was applied by the vane geometry on the sample for 6 min. 20 Pa was estimated to correlate to the typical gravitational stress acting on toothpaste in an average size toothpaste tube, when the tube is placed horizontally and left on the counter. During the 6 min of stress application, i.e. the creep step, the rotation of the vane was monitored, and the instantaneous viscosity was calculated. The instantaneous viscosity was the applied stress divided by the instantaneous shear rate. The instantaneous viscosity was plotted vs. Creep step time, as in FIG. 1 and FIG. 2.

### Viscosity Recovery

TABLE 1 shows the viscosity recovery of several example formulations as provided by the Creep Test described herein. The toothpaste compositions used in TABLE 1 were identical except for the amount of surface active polymer, polyvinylpyrrolidine (PVP) in this example. Note the estimated BET surface area for Z165 thickening silica was 120 m²/g for calculations in TABLE 1. However, the surface area can be considerably higher, upwards of 650 m²/g. The toothpaste composition included glycerin, polyethylene glycol, polyphosphates, sweeteners, other thickeners, surfactants, buffers, flavors and aesthetics commonly found in marketed toothpaste.

TABLE 1
Example toothpaste composition with 10.5
wt % silica (Z165, thickening silica)

| Surface | | Estimated Surface | Viscosity Increase from |
|---|---|---|---|
| Active Polymer | | Active Polymer/Silica | 1 to 348 sec at 20 Pa |
| (wt %) | | (mg/m²) | stress step (multiple) |
| 0% | PVP | 0 | 1.0 X |
| 0.072% | PVP | 0.057 | 111.5 X |
| 0.14% | PVP | 0.111 | 265.0 X |
| 0.3% | PVP | 0.238 | 767.3 X |
| 0.72% | PVP | 0.571 | 194.0 X |
| 2% | PVP | 1.587 | 6.0 X |

As shown in TABLE 1, without a surface active polymer present, such as PVP, there is almost no viscosity increase in the toothpaste composition after 5 minutes. However, upon the addition of as little as 0.072 wt % of PVP, corresponding to a surface active polymer/silica surface area ratio of 0.057 mg/m², led to a viscosity increase of 111.5×. This unexpected result of a unique viscosity increase can lead to toothpaste compositions that will not leak from the tube after dispensing because the viscosity can quickly increase after the dispensing process is completed.

The addition of PVP at 0.14 wt %, 0.3 wt %, and 0.72 wt % also had very high viscosity increases in as little as 5 minutes after the shear event (265×, 767×, and 194×, respectively). However, at wt % of PVP of 2 wt %, the effect (6× increase in viscosity), while still present, was less apparent. The compositions in TABLE 1 included thickening silica (Z165) at 10.5 wt % so the amount of surface active polymer needed can be tailored to the total surface area available. Thickening silica has more surface area available to be associated with surface active polymer. Thus, the amount of surface active polymer can be tailored based on the median particle size and amount of silica particles present in the toothpaste composition. FIG. 1 visually displays the viscosity recovery of TABLE 1.

TABLE 2 shows the viscosity recovery of several example formulations as provided by the Creep Test described herein. The toothpaste compositions used in TABLE 2 were identical except for the amount of surface active polymer, polyvinylpyrrolidine (PVP) in this example. TABLE 2 used 25 wt % of abrasive silica (Z109).

TABLE 2
Example toothpaste composition with
25 wt % abrasive silica (Z109)

| | | |
|---|---|---|
| Surface | Surface Active | Viscosity Increase from |
| Active Polymer | Polymer/Silica | 1 to 348 sec at 20 Pa |
| (wt %) | (mg/m²) | stress step (%) |

| | | | |
|---|---|---|---|
| 0% | PVP | 0 | 1.1 X |
| 0.05% | PVP | 0.083 | 4.1 X |
| 0.1% | PVP | 0.167 | 35.3 X |
| 0.5% | PVP | 0.833 | 1.7 X |
| 1% | PVP | 1.667 | 1.5 X |
| 1.5% | PVP | 2.500 | 1.2 X |
| 2.0% | PVP | 3.333 | 1.2 X |

As shown in TABLE 2, without a surface active polymer present, such as PVP, there is almost no viscosity increase in the toothpaste composition after 5 minutes (0.0% PVP in TABLE 2). However, upon the addition of as little as 0.05 wt % of PVP, corresponding to a surface active polymer/silica surface area ratio of 0.083 mg/m², led to a viscosity increase of 4.1×. Additionally, the addition of 0.10 wt % of PVP, corresponding to a surface active polymer/silica surface area ratio of 0.167 mg/m², led to a viscosity increase of 35.3×. These unexpected results of a unique viscosity increase can lead to toothpaste compositions that will not leak from the tube after dispensing because the viscosity can quickly increase after the dispensing process is completed.

The addition of PVP at 0.5 wt % and 1.0 wt % also had viscosity increases in as little as 348 seconds after the shear event. However, at wt % of PVP of 1.5 wt % and 2.0 wt %, the effect, while still present, was less apparent. The compositions in TABLE 2 included abrasive silica (Z109) at 25 wt %. Thus, the amount of surface active polymer needed can be tailored to the total surface area available. Abrasive silica has less surface area available to be associated with surface active polymer. Thus, the amount of surface active polymer can be tailored based on the median particle size and amount of silica particles present in the toothpaste composition. FIG. 2 visually displays the viscosity recovery of TABLE 2.

In total, it is desirable for a toothpaste formulation to have the ability to rapidly recover the yield stress and/or viscosity after a shear event such as the dispensing. Having this rapid viscosity recovery will aid in preventing leakage from the tube. As shown in FIG. 1 and FIG. 2, the curves with PVP added show an increase in viscosity and thixotropy compared to the control with no PVP added. The various levels of PVP and PVP to silica surface areas are further illustrated in TABLE 1 and 2.

In total, desirable ratios of mg surface active polymer to surface area of silica include from about 0.01 to about 3, from about 0.01 to about 2, from about 0.01 to about 1.5, from about 0.01 to about 1, from about 0.05 to about 0.8, from about 0.05 to about 0.6, or from about 0.1 to about 0.5 mg/m².

Additionally, desirable compositions have a first and a second viscosity and the second viscosity is at least about 1.5×, at least about 2×, at least about 3×, at least about 4×, at least about 5×, at least about 10×, from about 2× to about 1500×, from about 3× to about 1250×, or from about 5× to about 1000× greater than the first viscosity when the compositions are subjected to the Creep Test, as described herein. “X” as used herein means times, e.g. “2×” means twice or two times and “1000×” means one thousand times. In other words, the “X” indicates an x-fold increase in viscosity of the second viscosity relative to the first viscosity.

The first viscosity of desirable toothpaste compositions can be measured less than about 10 seconds, less than about 5 seconds, less than about 1 second, or immediately after a shear event, such as dispensing the toothpaste composition from a conventional toothpaste tube.

The second viscosity of desirable toothpaste compositions can be measured at least about 50 seconds, at least about 100 seconds, at least about 200 seconds, at least about 250 seconds, at least about 350 seconds, or approximately 348 seconds after the shear event, such as dispensing the toothpaste composition from a conventional toothpaste tube, as described above.

These unexpected results were observed with both abrasive silica, such as Z109, and thickening silica, such as Z165. Thus, it is expected that any silica would lead to these unexpected results. Additionally, it is expected that any surface active polymer that can bridge between silica particles can exhibit these results.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A toothpaste composition comprising:
(a) from about 0.01% to about 5%, by weight of the composition, of surface active polymer;
(b) from about 0.1% to about 40%, by weight of the composition of abrasive, the abrasive comprising silica; and
(c) less than 10%, by weight of the composition, of water, wherein the toothpaste composition has a ratio of surface active polymer to silica surface area of from about 0.01 to about 2.0 mg/m².

2. The toothpaste composition of claim 1, wherein the toothpaste composition comprises polyphosphate.

3. The toothpaste composition of claim 2, wherein the polyphosphate comprises pyrophosphate, tripolyphosphate, tetrapolyphopshate, sodaphospolyphosphate, hexaphospolyphosphate, benephospolyphosphate, hexametaphosphate, or combinations thereof.

4. The toothpaste composition of claim 1, wherein the toothpaste composition comprises no added water.

5. The toothpaste composition of claim 1, wherein the silica comprises silica particles with a median particle size of up to about 20 μm.

6. The toothpaste composition of claim 1, wherein the surface active polymer comprises polyvinylpyrrolidone, cross-linked polyvinylpyrrolidone, another polymer derived from a N-vinylpyrrolidone monomer, polyethylene oxide, poloxamer, or combinations thereof.

7. The toothpaste composition of claim 6, wherein the polyvinylpyrrolidone comprises only non-crosslinked polyvinylpyrrolidone.

8. The toothpaste composition of claim 1, wherein the toothpaste composition comprises whitening agent.

9. The toothpaste composition of claim 8, wherein the whitening agent comprises hydrogen peroxide, carbamide peroxide, or combinations thereof.

10. The toothpaste composition of claim 1, wherein the toothpaste composition comprises additional abrasive.

11. The toothpaste composition of claim 10, wherein the additional abrasive comprises calcium carbonate, calcium pyrophosphate, or combinations thereof.

12. The toothpaste composition of claim 1, wherein the toothpaste composition comprises fluoride.

13. The toothpaste composition of claim 12, wherein the fluoride comprises sodium fluoride, sodium monofluorophosphate, stannous fluoride, amine fluoride, or combinations thereof.

14. The toothpaste composition of claim 1, wherein the toothpaste composition comprises tin.

15. The toothpaste composition of claim 14, wherein the tin comprises stannous fluoride, stannous chloride, or combinations thereof.

16. The toothpaste composition of claim 1, wherein the toothpaste composition comprises zinc.

17. The toothpaste composition of claim 16, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.

18. The toothpaste composition of claim 1, wherein the ratio of surface active polymer to silica of from about 0.05 to about 0.8 mg/m².

19. The toothpaste composition of claim 1, wherein the toothpaste composition has a viscosity increase of at least about 300% based on the Creep Test.

20. A toothpaste composition comprising:
(a) from about 0.01% to about 5%, by weight of the composition, of surface active polymer;
(b) from about 0.1% to about 40%, by weight of the composition of abrasive, the abrasive comprising silica; and
(c) less than 10%, by weight of the composition, of water,
wherein the toothpaste composition has a first viscosity measured less than 5 seconds after a shear event and the toothpaste composition has a second viscosity measured at least 50 seconds after the shear event, and the second viscosity is greater the first viscosity.

21. The toothpaste composition of claim 20, wherein the second viscosity is at least about 1.5 times greater than the first viscosity.

22. The toothpaste composition of claim 21, wherein the composition comprises polyphosphate, the polyphosphate comprises pyrophosphate, tripolyphosphate, tetrapolyphopshate, sodaphospolyphosphate, hexaphospolyphosphate, benephospolyphosphate, hexametaphosphate, or combinations thereof.

23. The toothpaste composition of claim 20, wherein the toothpaste composition comprises no added water.

24. The toothpaste composition of claim 20, wherein the silica comprises silica particles with a median particle size of up to about 20 μm.

25. The toothpaste composition of claim 20, wherein the surface active polymer comprises polyvinylpyrrolidone, cross-linked polyvinylpyrrolidone, another polymer derived from a N-vinylpyrrolidone monomer, polyethylene oxide, poloxamer, or combinations thereof.

26. The toothpaste composition of claim 25, wherein the polyvinylpyrrolidone comprises only non-crosslinked polyvinylpyrrolidone.

27. The toothpaste composition of claim 20, wherein the toothpaste composition comprises whitening agent.

28. The toothpaste composition of claim 27, wherein the whitening agent comprises hydrogen peroxide, carbamide peroxide, or combinations thereof.

29. The toothpaste composition of claim 20, wherein the toothpaste composition comprises additional abrasive.

30. The toothpaste composition of claim 29, wherein the additional abrasive comprises calcium carbonate, calcium pyrophosphate, or combinations thereof.

31. The toothpaste composition of claim 20, wherein the toothpaste composition comprises fluoride.

32. The toothpaste composition of claim 31, wherein the fluoride comprises sodium fluoride, sodium monofluorophosphate, stannous fluoride, amine fluoride, or combinations thereof.

33. The toothpaste composition of claim 20, wherein the toothpaste composition comprises tin.

34. The toothpaste composition of claim 33, wherein the tin comprises stannous fluoride, stannous chloride, or combinations thereof.

35. The toothpaste composition of claim 20, wherein the toothpaste composition comprises zinc.

36. The toothpaste composition of claim 35, wherein the zinc comprises zinc fluoride, zinc lactate, zinc oxide, zinc phosphate, zinc chloride, zinc acetate, zinc hexafluorozirconate, zinc sulfate, zinc tartrate, zinc gluconate, zinc citrate, zinc malate, zinc glycinate, zinc pyrophosphate, zinc metaphosphate, zinc oxalate, zinc carbonate, or combinations thereof.

37. The toothpaste composition of claim 20, wherein the toothpaste composition has a ratio of surface active polymer to silica of from about 0.05 to about 0.8 mg/m².
